(19) United States
(12) Patent Application Publication
(10) Pub. No.: US 2010/0029774 A1
(43) Pub. Date: Feb. 4, 2010

(54) ALISKIREN MONOFUMARATE AND PROCESSES FOR PREPARATION THEREOF

(76) Inventors: Nina FINKELSTEIN, Herzliya (IL); Ariel Mittelman, Elad (IL)

Correspondence Address: KENYON & KENYON LLP, ONE BROADWAY, NEW YORK, NY, 10004, US

(21) Appl. No.: 12/470,766

(22) Filed: May 22, 2009

Related U.S. Application Data
(60) Provisional application No. 61/055,785, filed on May 23, 2008.

(51) Int. Cl. A61K 31/165; C07C 237/14; C07C 231/00

(52) U.S. Cl. 514/616; 564/157

(57) ABSTRACT

The present invention provides a novel fumarate compound of aliskiren monofumarate, and process for preparation thereof. The present invention also provides pharmaceutical compositions comprising aliskiren monofumarate, and methods of using aliskiren monofumarate for treating hypertension.

### RELATED APPLICATION

This application claims the benefit under 35 U.S.C. §119(e) of U.S. Provisional Application No. 61/055,785, filed May 23, 2008, which is incorporated herein by reference in its entirety.

### FIELD OF THE INVENTION

The present invention relates to a novel monofumarate compound of aliskiren, and a process for preparing said compound.

### BACKGROUND OF THE INVENTION

Aliskiren hemifumarate [CAS Registry Number: 173334-58-2], having the chemical name: (2S, 4S, 5S, 7S)-N-(2-carbamoyl-2-methylpropyl)-5-amino-4-hydroxy-2,7-diisopropyl-8-[4-methoxy-3-(3-methoxypropoxy)phenyl]octanamide hemifumarate [C₃₀H₅₃N₃O₆.0.5 C₄H₄O₄] and the following structure:

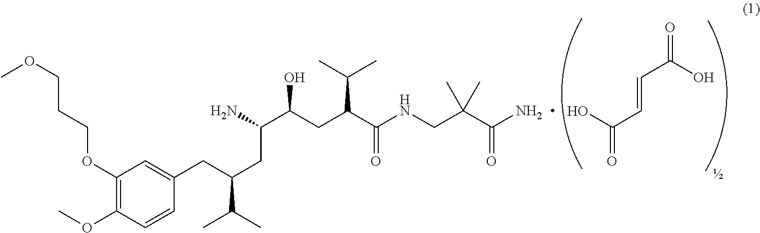

is indicated for treatment of hypertension, acting as a renin inhibitor, and marketed by Novartis as TEKTURNA® in a once-daily formulation. Synthesis of aliskiren and its related compounds are referred to in U.S. Pat. No. 5,559,111, while pharmacological actions, pharmacokinetics and clinical studies of aliskiren and its related compounds are referred to in Lindsay, K. B. et al., J. Org. Chem., Vol. 71, pp 4766-4777 (2006) and in Drugs of the Future, Vol. 26, No. 12, pp 1139-1148 (2001).

U.S. Pat. No. 5,559,111 refers to the preparation of a crystalline form of aliskiren hemifumarate having a melting point of about 95-104° C. by crystallizing from an ethanol/acetonitrile mixture in a 1 to 19 volume ratio and then drying at 60° C.

U.S. Pat. No. 6,730,798 refers to the preparation of aliskiren hemifumarate from aliskiren base and fumaric acid in ethanol/acetonitrile. U.S. Publication No. 2006/0154926 (US'926) describes the preparation of aliskiren hydrochloride. Preparation of aliskiren hemifumarate from aliskiren hydrochloride is also described in US'926.

Publication No. WO2007/107317 describes the preparation of crystalline aliskiren hydrogen-sulfate.

Publication No. WO2007/098503 describes the preparation of crystalline aliskiren nitrate.

Publication No. WO2008/055669 describes the preparation of crystalline aliskiren orotate.

The discovery of new forms of a pharmaceutically useful compound provides an opportunity to improve the performance characteristics of a pharmaceutical product. It enlarges the repertoire of materials that a formulation scientist has available for designing, for example, a pharmaceutical dosage form of a drug with a targeted release profile or other desired characteristics. There is a need in the art for new forms of pharmaceutically useful compounds of aliskiren.

### SUMMARY OF THE INVENTION

The present invention encompasses aliskiren monofumarate. In one embodiment, the invention provides an isolated monofumarate compound of aliskiren, preferably in a solid form.

The present invention further provides an amorphous form of said monofumarate compound of aliskiren.

The present invention also provides a process for preparing aliskiren monofumarate comprising providing a first solution of aliskiren hemifumarate and fumaric acid in a C₁-C₄ alcohol; removing the solvent to obtain a solid; combining the solid with an acetonitrile/C₁-C₄ alcohol mixture to obtain a second solution; and further removing the solvents from the second solution to obtain the aliskiren monofumarate. The aliskiren monofumarate obtained according to the process of the present invention is preferably in an amorphous form.

The present invention further encompasses 1) a pharmaceutical composition comprising the aliskiren monofumarate described above and at least one pharmaceutically acceptable excipient, and 2) the use of the above-described aliskiren monofumarate for the manufacture of a pharmaceutical composition, wherein the pharmaceutical composition can be useful for the treatment of hypertension.

The pharmaceutical composition of the present invention can be in a solid or a non-solid form. If the pharmaceutical composition is in a non-solid form, the aliskiren monofumarate in the composition can present as a solid in the non-solid pharmaceutical composition, e.g., as a suspension, foam, ointment, etc.

The pharmaceutical composition can be prepared by a process comprising combining the above-described aliskiren monofumarate with at least one pharmaceutically acceptable excipient. The aliskiren monofumarate can be obtained by any of the processes of the present invention as described herein.

The pharmaceutical composition can be used to make appropriate dosage forms such as tablets, powders, capsules, suppositories, sachets, troches and lozenges.

The aliskiren monofumarate of the present invention, particularly in a pharmaceutical composition and dosage form, can be used to treat hypertension in a mammal such as a human by administering a treatment effective amount of the aliskiren monofumarate to the mammal. The treatment effective amount or proper dosage to be used can be determined by one of ordinary skill in the art, which can depend on the method of administration, the bioavailability, the age, sex, symptoms and health condition of the patient, and the severity of the disease to be treated, etc.

The aliskiren monofumarate used in any of the above-described pharmaceutical compositions is preferably in a solid form and most preferably in an amorphous form.

### BRIEF DESCRIPTION OF THE FIGURES

FIG. 1 shows a powder XRD pattern of amorphous aliskiren monofumarate.

### DETAILED DESCRIPTION OF THE INVENTION

The solid state physical properties of an active pharmaceutical ingredient (API), such as aliskiren, affect the commercial usefulness of the API. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid may have therapeutic consequences since it imposes an upper limit on the rate at which an orally-administered active ingredient may reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which define a particular form of a substance. An amorphous form may have thermal behavior different from that of a polymorphic form. A particular form may also give rise to distinct spectroscopic properties that may be detectable by powder X-ray crystallography, solid state C NMR spectrometry and infrared spectrometry. The solid state physical properties of aliskiren may be influenced by controlling the conditions under which it is obtained in solid form.

The present invention provides a solid form of an aliskiren monofumarate compound with increased solubility in water as compared to the aliskiren free base. Increased solubility leads to improved bioavailability when the drug is administered to a patient, and, thus, allows reduced required dosages. One embodiment of the invention is an amorphous form of aliskiren monofumarate, which is more readily soluble than aliskiren free base.

As used herein, unless otherwise defined, the term “aliskiren monofumarate” refers to an aliskiren compound, in which aliskiren base and fumaric acid are present in a molar ratio of about 1:1.

As used herein, “room temperature” refers to a temperature of about 15° C. to about 30° C.

As used herein, “isolated” refers to a compound being physically separated from the reaction mixture. For example, the separation can be done by elution from an HPLC column and further drying the compound. Preferably aliskiren monofumarate according to one embodiment of the present invention contains less than 1%, more preferably less than 0.5% and most preferably is substantially free (e.g. less than 0.05%) of aliskiren free base. The aliskiren monofumarate according to one embodiment of the present invention preferably contains less than 1%, more preferably less than 0.5% and most preferably is substantially free (e.g., less than 0.05%) of aliskiren hemifumarate. Preferably the aliskiren monofumarate according to the invention contains less than 0.5% of aliskiren hemifumarate and less than 0.5% aliskiren free base, and is more preferably substantially free (e.g., less than 0.05%) of both free base and hemifumarate forms. In any of these embodiments, the aliskiren monofumarate is preferably in amorphous form as described in any of the embodiments below.

As used herein, “reduced pressure” refers to a pressure of below atmospheric pressure, i.e., a pressure of less than 1 atm.

Aliskiren monofumarate may be analyzed to determine the nature of the product. The X-ray powder diffraction pattern of amorphous aliskiren monofumarate does not exhibit peaks characteristic of crystal forms of aliskiren monofumarate, demonstrating the amorphous nature of the product. The presence of characteristic peaks for crystalline forms would indicate the presence of a crystalline form of aliskiren monofumarate.

In one embodiment, the invention provides aliskiren monofumarate.

The aliskiren monofumarate of the present invention can be represented by the chemical name (2S, 4S, 5S, 7S)-N-(2-carbamoyl-2-methylpropyl)-5-amino-4-hydroxy-2,7-diisopropyl-8-[4-methoxy-3-(3-methoxypropoxy)phenyl]octanamide monofumarate and by the following molecular structure:

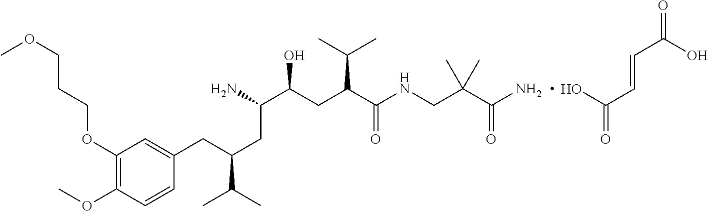

In another embodiment, the present invention provides isolated aliskiren monofumarate.

In yet another embodiment, the present invention provides a solid aliskiren monofumarate.

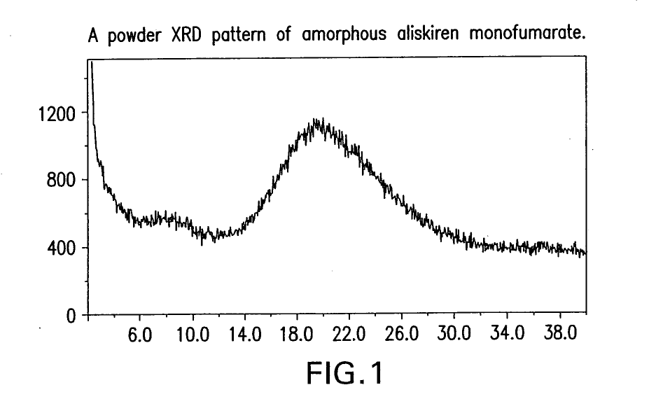

In one embodiment, the invention provides an amorphous aliskiren monofumarate as characterized by the X-ray powder diffraction pattern depicted in FIG. 1.

The aliskiren monofumarate may be prepared by a process comprising providing a first solution of aliskiren hemifumarate and fumaric acid in a C₁-C₄ alcohol; removing the solvent to obtain a solid; combining the solid with an acetonitrile/C₁-C₄ alcohol mixture to obtain a second solution; and further removing the solvents from the second solution to obtain the aliskiren monofumarate. Preferably the solvent is removed by evaporation, more preferably by evaporation under reduced pressure.

The aliskiren monofumarate obtained according to the process described above is preferably in an amorphous form.

The aliskiren hemifumarate starting material can be prepared by any method known in the art such as the one described in U.S. Pat. Nos. 6,730,798 and 5,559,111 incorporated herein by reference. Also, the aliskiren hemifumarate starting material can be in any crystalline form or the amorphous form.

Preferably, the first solution is obtained by combining aliskiren hemifumarate, fumaric acid and methanol. The molar ratio between fumaric acid and aliskiren hemifumate used in the first solvent is preferably from 1:2 fumaric acid to aliskiren hemifumarate. Preferably the molar ratio of fumaric acid to aliskiren hemifumarate is about 1:2 to 1.5:2, more preferably about 1:2 to about 1.2:2, or 1:2 to about 1.1:2, and most preferably about 1.05:2 to about 1:2. Particularly preferred is a molar ratio of fumaric acid to aliskiren hemifumarate of about 1:2.

The first solution can be obtained at about room temperature to about the reflux temperature of the solvent. Preferably, it is obtained at about room temperature. More preferably, it is obtained at about 15° C. to about 25° C., and even more preferably at about 20° C. to about 25° C.

The acetonitrile/C₁-C₄ alcohol mixture of the second solution is preferably in a ratio of about 80:20 to about 98:2 (v/v) acetonitrile to C₁-C₄ alcohol. More preferably, the ratio is about 90:10 to about 98:2 (v/v)., and even more preferably the ratio is about 95:5 (v/v). The C₁-C₄ alcohol used in the second solution is preferably methanol, ethanol or isopropyl alcohol (IPA). More preferably, the alcohol is methanol or ethanol and most preferably ethanol is used.

Alternatively, the first solution may be obtained by combining aliskiren free base, fumaric acid and methanol. The molar ratio between fumaric acid and aliskiren free base used in the first solvent is preferably from 1:1 fumaric acid to aliskiren free base. Preferably the molar ratio of fumaric acid to aliskiren free base is about 1:1 to about 1.5:1, more preferably about 1:1 to about 1.2:1, or 1:1 to about 1.1:1, and most preferably about 1:1 to about 1.05:1. Particularly preferred is a molar ratio of fumaric acid to aliskiren free base of about 1:1.

The aliskiren free base starting material can be obtained by any method known in the art, such as, for example, U.S. Pat. No. 6,730,798 and U.S. Pat. No. 5,559,111.

Removal of the solvent can be done by any conventional method, such as evaporating the solvents. Preferably, evaporating is performed under reduced pressure.

Amorphous aliskiren hemifumarate used in the process described above may be obtained according to any of the methods described in International Application No. PCT/US2008/012816, filed on Nov. 13, 2008.

The present invention further encompasses 1) a pharmaceutical composition comprising the aliskiren monofumarate described above and at least one pharmaceutically acceptable excipient, and 2) the use of the above-described aliskiren monofumarate, for the manufacture of a pharmaceutical composition, wherein the pharmaceutical composition can be useful for the treatment of hypertension.

The pharmaceutical composition of the present invention can be in a solid or a non-solid form. If the pharmaceutical composition is in a non-solid form, the aliskiren monofumarate in the composition can present as a solid in the non-solid pharmaceutical composition, e.g., as a suspension, foam, ointment, etc.

The pharmaceutical composition can be prepared by a process comprising combining the above-described aliskiren monofumarate with at least one pharmaceutically acceptable excipient. The aliskiren monofumarate can be obtained by any of the processes of the present invention as described above.

The pharmaceutical composition can be used to make appropriate dosage forms such as tablets, powders, capsules, suppositories, sachets, troches and lozenges.

The aliskiren monofumarate of the present invention, particularly in a pharmaceutical composition and dosage form, can be used to treat hypertension in a mammal such as a human, comprising administering a treatment effective amount of the aliskiren monofumarate in the mammal. The treatment effective amount or proper dosage to be used can be determined by one of ordinary skill in the art, which can depend on the method of administration, the bioavailability, the age, sex, symptoms and health condition of the patient, and the severity of the disease to be treated, etc.

The aliskiren monofumarate used in any of the above-described pharmaceutical compositions is preferably in a solid form and most preferably in an amorphous form.

Having thus described the invention with reference to particular preferred embodiments and illustrative examples, those in the art can appreciate modifications to the invention as described and illustrated that do not depart from the spirit and scope of the invention as disclosed in the specification. The examples are set forth to aid in understanding the invention but are not intended to, and should not be construed to, limit its scope in any way.

### EXAMPLES

### Powder XRD (X-Ray Diffraction)

An ARL X-ray powder diffractometer, model X`TRA-030, with a Peltier detector and a round standard aluminum sample holder with a round zero background silicon plate was used. The cathode is CuKa radiation; λ=1.5418 Å. Scanning parameters: range: 2-40 deg. 2, continuous scans, rate: 3 deg/min. The accuracy of peak positions is defined as +/−0.2 degrees due to experimental differences such as instrumentation and sample preparation.

### Example 1

Aliskiren hemifumarate amorphous (300 mg, 0.5 mmol) and fumaric acid (29 mg, 0.25 mmol) were dissolved in 5 ml of methanol, by stirring at room temperature. After evaporation of methanol under vacuum, the product was dissolved in an acetonitrile/ethanol mixture (95:5) (5 ml), and the solvents were evaporated under vacuum.

What is claimed is:

1. Aliskiren monofumarate, (2S, 4S, 5S, 7S)-N-(2-carbamoyl-2-methylpropyl)-5-amino-4-hydroxy-2,7-diisopropyl-8-[4-methoxy-3 -(3-methoxypropoxy)phenyl] octanamide monofumarate, having the following formula:

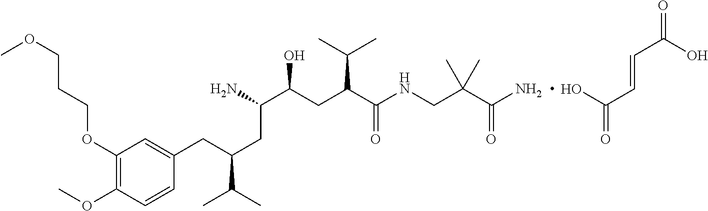

2. The aliskiren monofumarate of claim 1, which is isolated.

3. The aliskiren monofumarate of claim 1 or claim 2, which is a solid.

4. The aliskiren monofumarate of claim 1 or claim 2, which is in amorphous form.

5. The aliskiren monofumarate of claim 4, characterized by an X-ray powder diffraction pattern depicted in FIG. 1.

6. A process for preparing aliskiren monofumarate, comprising:
(a) providing a first solution of aliskiren hemifumarate and fumaric acid in a C₁-C₄ alcohol;
(b) removing the solvent to obtain a solid;
(c) combining the solid of step (b) with an acetonitrile/C₁-C₄ alcohol mixture to obtain a second solution; and
(d) further removing the solvents from the second solution of step (c) to obtain the aliskiren monofumarate.

7. The process of claim 6, wherein the obtained aliskiren monofumarate is in amorphous form.

8. The process of claim 6 or claim 7, wherein the fumaric acid and aliskiren hemifumarate in step (a) are in a molar ratio of 1:2 of fumaric acid to aliskiren hemifumarate.

9. The process of claim 6 or claim 7, wherein the C₁-C₄ alcohol in step (a) is methanol.

10. The process of claim 6 or claim 7, wherein the first solution is obtained at about room temperature to about reflux.

11. The process of claim 6 or claim 7, wherein the acetonitrile/C₁-C₄ alcohol mixture in step (c) is at a ratio of about 80:20 to about 98:2 (v/v) of acetonitrile to C₁-C₄ alcohol.

12. The process of claim 6 or claim 7, wherein the acetonitrile/C₁-C₄ alcohol mixture in step (c) is at a ratio of 95:5 (v/v) of acetonitrile to C₁-C₄ alcohol.

13. The process of claim 6 or claim 7, wherein the C₁-C₄ alcohol in step (c) is ethanol, methanol or isopropyl alcohol.

14. The process of claim 13, wherein the C₁-C₄ alcohol in step (c) is ethanol.

15. The process of claim 6 or claim 7, wherein the solvent is removed by evaporation.

16. The process of claim 15, wherein evaporation is performed under reduced pressure.

17. A pharmaceutical composition comprising the aliskiren monofumarate of claim 1 or claim 2 and at least one pharmaceutically acceptable excipient.

18. A pharmaceutical composition according to claim 17, wherein the aliskiren monofumarate is in amorphous form.

19. A process for preparing a pharmaceutical composition comprising aliskiren monofumarate, comprising combining the aliskiren monofumarate of claim 1 or 2 with at least one pharmaceutically acceptable excipient.

20. The process according to claim 19, wherein the aliskiren monofumarate is in amorphous form.

21. A process for preparing a pharmaceutical composition comprising aliskiren monofumarate, comprising combining the aliskiren monofumarate produced by the process of claim 6 or claim 7 with at least one pharmaceutically acceptable excipient.
